(19) United States
(12) Patent Application Publication
(10) Pub. No.: US 2024/0115813 A1
(43) Pub. Date: Apr. 11, 2024

(54) Nozzle Holder

(71) Applicant: Merxin Ltd, King's Lynn (GB)

(72) Inventor: Adam Stuart, King's Lynn (GB)

(21) Appl. No.: 18/045,187

(22) Filed: Oct. 10, 2022

(51) Int. Cl. A61M 15/00; B22F 1/10; B22F 3/00; B22F 3/11

(52) U.S. Cl. CPC A61M 15/0001; B22F 1/10; B22F 3/004; B22F 3/1103; B22F 2301/35; B22F 2998/10

(57) ABSTRACT

In general terms the present invention proposes a nozzle holder for securing a nozzle in an inhalation device, for example such as a soft mist inhaler (SMI), the nozzle holder being obtained or obtainable by metal injection moulding (MIM). Also disclosed is an inhalation device comprising the nozzle holder.

### TECHNICAL FIELD

This invention relates to a nozzle holder for securing a nozzle in an inhalation device. In particular, though not exclusively, this invention relates to a nozzle holder formed by metal injection moulding (MIM), and to an inhalation device comprising the nozzle holder.

### BACKGROUND

Drug delivery devices such as soft mist inhalers (SMIs) can be used to produce an aerosol of droplets for inhalation through the mouth and pharyngeal cavity into the lungs of a patient, for nasal administration, or for spraying the surface of the eye.

In an inhaler of this kind, liquid pharmaceutical formulations are typically stored in a reservoir. From there, they are conveyed through a riser tube into a pressure chamber from where they are forced through a nozzle under pressure and atomised. In this way, SMIs are able to nebulise a small amount of a liquid formulation according to the required dosage within a few seconds to produce an aerosol suitable for therapeutic inhalation. Moreover, this can be achieved without requiring the use of a propellant.

The nozzle is typically held in place in the device by a nozzle holder. This nozzle holder itself may be secured in the device by a fixing device. As the liquid formulation is forced through the nozzle under pressure, a small amount of the liquid may be deposited as a film or as an accumulation of small droplets on the surface of the nozzle holder. It has been found that the deposited liquid can disrupt the flow of further liquid through the nozzle, which can affect the pharmaceutical quality of the aerosol mist.

Hence, there remains a need for improved drug delivery devices that can control the proportion and location/distribution of liquid deposited on the surface of the nozzle holder. It is an object of the invention to address at least one of the above problems, or another problem associated with the prior art.

### SUMMARY OF THE INVENTION

A first aspect of the invention provides a nozzle holder for securing a nozzle in an inhalation device, the nozzle holder being obtained or obtainable by metal injection moulding (MIM).

Such a nozzle holder has advantageously been found to exhibit a surface finish that reduces the formation of droplets on the surface of the nozzle holder, thereby minimising or preventing disruption of the flow of further liquid through the nozzle. Significantly, this may allow for greater consistency of drug delivery from an inhalation device comprising such a nozzle holder through improved retained droplet control.

A “nozzle holder” as defined herein is a component suitable for holding or securing a nozzle in place in an inhalation device. Suitably, the nozzle holder may comprise an aperture through which a liquid spray may be delivered from the nozzle through the nozzle holder. Suitably, a first side of the nozzle holder may comprise a first recess for receiving or accommodating at least a portion of the nozzle. Where the inhalation device comprises a nozzle seal that at least partially surrounds the nozzle, the first recess may also receive or accommodate at least a portion of the nozzle seal. Suitably, a second side of the nozzle may comprise a second recess for directing delivery of a liquid spray delivered from the nozzle through nozzle holder, for example through the aperture of the nozzle holder.

A “nozzle” as defined herein is a component for nebulising (i.e. generating a spray of fine droplets from) a liquid. The nozzle may suitably comprise a nozzle chip. A “nozzle chip” as defined herein is a component having an inlet end and an outlet end connected by a plurality of microstructured channels. The inlet end of the nozzle chip may comprise a filtering structure, comprising one or more microstructured channels. In this way, the filtering structure may advantageously prevent any coarse debris from blocking the microstructured channels at the outlet end. The outlet end of the nozzle chip may comprise one or more spray jets. Where two or more spray jets are present, the geometries of the spray jets may be suitably be arranged to cause two or more jets of liquid exiting the spray jets to impinge upon one another (i.e. collide with each other).

The inhalation device may be a device suitable for dispensing a liquid, such as a pharmaceutical liquid, in the form of an aerosol from a reservoir or canister comprising the liquid. The term “pharmaceutical liquid” as defined herein refers to a solution, emulsion, or suspension of one or more active pharmaceutical ingredients in a suitable solvent. The inhalation device may be an inhaler for nebulising pharmaceutical liquids. For example, the inhalation device may be a soft mist inhaler (SMI).

In some embodiments, the nozzle holder may comprise an exterior surface having a water contact angle (θ) equal to or less than 60°.

For example, the nozzle holder may comprise an exterior surface having a water contact angle (θ) equal to or less than 58°, or equal to or less than 56°, or equal to or less than 54°, or equal to or less than 52°, or equal to or less than 50°, or equal to or less than 48°, or equal to or less than 46°, or equal to or less than 44°, or equal to or less than 42°, or equal to or less than 40°, equal to or less than 38°, or equal to or less than 36°, or equal to or less than 34°, or equal to or less than 32°, or equal to or less than 30°, or equal to or less than 28°, or equal to or less than 26°, or equal to or less than 24°, or equal to or less than 22°, or equal to or less than 20°, or equal to or less than 18°, or equal to or less than 16°, or equal to or less than 14°, or equal to or less than 12°, such as equal to or less than 10°.

In some embodiments, the nozzle holder may comprise an exterior surface having a water contact angle (θ) equal to or less than 90°, or equal to or less than 85°, equal to or less than 80°, equal to or less than 75°, equal to or less than 70°, such as equal to or less than 65°.

In some embodiments, the nozzle holder may comprise an exterior surface having a surface roughness average (Ra) of 0.56 microns or more.

For example, the nozzle holder may comprise an exterior surface having a surface roughness average (Ra) of 0.63 microns or more, or of 0.7 microns or more, or of 0.8 microns or more, or of 0.9 microns or more, or of 1 micron or more, such as of 1.12 microns or more.

In some embodiments, the exterior surface of the nozzle holder may have a non-periodic surface texture. For example, the surface texture may be random without any repeating surface features.

In some embodiments, 40% or more of the external surface of the nozzle holder may be pocketed or cavitied. For example, 50% or more, or 60% or more, or 70% or more, or 80% or more, or even 90% or more of the external surface of nozzle holder may be pocketed or cavitied.

Suitably, the nozzle holder may be non-rotationally symmetric.

In some embodiments, the nozzle holder may comprise a non-rotationally symmetric recess. For example, the nozzle holder may comprise an oblong, elliptical or oval shaped recess.

In some embodiments, the nozzle holder may comprise or be formed from a metal. Suitably the metal may comprise steel, for example such as stainless steel.

A second aspect of the invention provides an inhalation device comprising a nozzle holder rotationally symmetric nozzle holder according to the first aspect of the invention.

A third aspect of the invention provides a method of making a nozzle holder, for example a non-rotationally symmetric nozzle holder, the method comprising the steps of:
mixing metal powder with a polymer binder;
injecting the mixture into a mould with an extruder;
debinding; and
sintering.

In some embodiments, the metal powder may comprise a metal. Suitably the metal may comprise steel, for example such as stainless steel.

In some embodiments, the polymer binder may comprise one or more of polyethylene glycol, polyethylene oxide, polyvinyl alcohol, starches, and/or polyacrylamide.

In some embodiments, the step of debinding may comprise one or more of solvent debinding, thermal debinding, catalytic debinding, and/or supercritical debinding.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is a perspective view of a MIM nozzle holder;

FIG. 2 is a cross-sectional view of a MIM nozzle holder in a nozzle fixing assembly;

FIG. 3 is a cross-sectional view of a MIM nozzle holder in an alternative nozzle fixing assembly;

FIGS. 4(a) to (d) are images of the surface of a MIM nozzle holder; and

FIGS. 5(a) to (d) are images of the surface of a turned nozzle holder.

### DETAILED DESCRIPTION

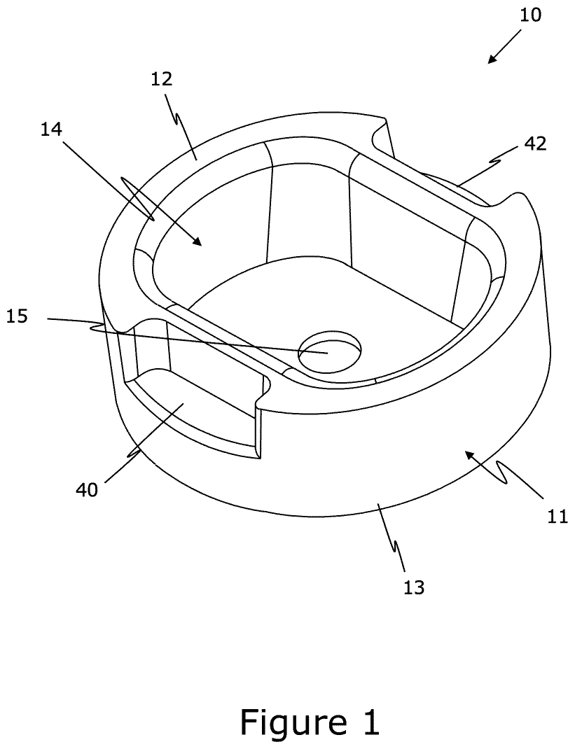

Referring to FIG. 1, a stainless steel MIM nozzle holder 10 according to a first embodiment of the invention is rotationally non-symmetric, defining a cylindrical body 11 having first and second ends 12, 13. The first end 12 of the nozzle holder comprises a first oblong-shaped recess 14 for receiving a nozzle (not shown). An aperture 15 is located at the centre of the first recess 14 and provides a passage through the nozzle holder 10 through which a liquid spray from the nozzle may be delivered from the first end 12 to the second end 13. The second end 13 of the nozzle holder 10 comprises a second cone-shaped projection 16 (not visible in FIG. 1), which narrows down to the aperture 15. The second recess directs liquid spray from the nozzle away from the aperture 15 out of the second end 13 of the nozzle holder 10. The MIM nozzle holder 10 also comprises two rotational alignment features 40, 42, which define stepped recesses on opposite sides of the first end 12.

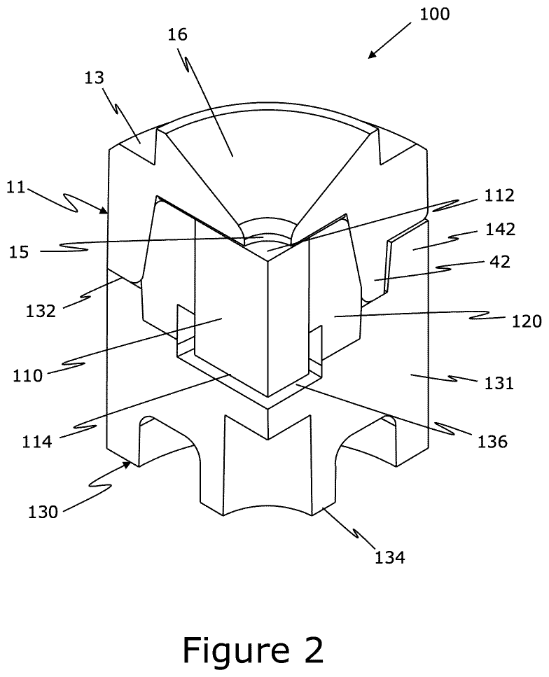

FIG. 2 illustrates a nozzle fixing assembly 100 for an inhalation device comprising the stainless steel MIM nozzle holder 10. The nozzle fixing 100 assembly also comprises a nozzle chip 110, nozzle seal 120 and filter holder 130. The nozzle chip 110 is a cuboidal in shape, having an elongate length between opposing first and second ends 112, 114. The nozzle seal 120 surrounds the nozzle chip 110 and extends about two thirds the way along its elongate length from the first end 112 of the nozzle chip 110 towards the second end 114 of the nozzle chip 112. The first end 12 of the nozzle holder receives the first the end 112 of the nozzle chip 110 and the nozzle seal 120.

The filter holder 130 defines a generally cylindrical body 131 having first and second ends 132, 134. The first end 132 of the filter holder 130 abuts the first end 12 of the nozzle holder and comprises a cylindrical-shaped recess 136 for receiving the nozzle chip 110. In this way, the recess 136 of the filter holder 130 and the first recess 14 of the nozzle holder 10 together form an enclosed volume in which the nozzle chip 110 and nozzle seal 120 are contained. The nozzle holder 10 therefore secures the nozzle chip 110 and nozzle seal 120 in place with respect to the filter holder 130 to form the fixing assembly 100. The fixing assembly 100 may itself secured in place in the inhaler device by a check nut (not shown).

The filter holder 130 also comprises two rotational alignment features 140 (not visible in FIG. 2) and 142, which define projections extending from the first end 132 of the filter holder 130. The rotational alignment features 140, 142 are arranged to interlock with the two rotational alignment features 40, 42 on opposite sides of the first end 12 of the MIM nozzle holder 10 to prevent relative rotation of the MIM nozzle holder 10 with respect to the filter holder 130. The restriction of this relative rotation prevents any twisting forces being applied to the nozzle seal 120 and ensures that the nozzle chip 110 is aligned to the oblong-shaped recess 14 of the MIM nozzle holder 10 and cannot adversely contact the filter holder 130.

FIG. 2 illustrates an alternative nozzle fixing assembly 200 for an inhalation device comprising the stainless steel MIM nozzle holder 10. The nozzle fixing 200 assembly also comprises a nozzle chip 210, nozzle seal 220, rubber O-ring 221, and filter holder 230. The nozzle chip 210 is a cuboidal in shape, having an elongate length between opposing first and second ends 212, 214. The nozzle seal 220 surrounds the nozzle chip 210 and extends about half way along its elongate length from the first end 212 of the nozzle chip 210 towards the second end 214 of the nozzle chip 212. The rubber O-ring 221 surrounds the remaining portion of the nozzle chip 210 and abuts the nozzle seal 220. The first end 12 of the nozzle holder receives the first the end 212 of the nozzle chip 110 and the nozzle seal 120.

The filter holder 230 defines a generally cylindrical body 231 having first and second ends 232, 234. The first end 232 of the filter holder 230 abuts the first end 12 of the nozzle holder and comprises a cylindrical-shaped recess 236 for receiving the nozzle chip 210 and rubber O-ring 221. In this way, the recess 236 of the filter holder 230 and the first recess 14 of the nozzle holder 10 together form an enclosed volume in which the nozzle chip 210, nozzle seal 220, and rubber O-ring 221 are contained. The nozzle holder 10 therefore secures the nozzle chip 210, nozzle seal 220, and rubber O-ring 221 in place with respect to the filter holder 230 to form the fixing assembly 200. The fixing assembly 200 may itself secured in place in the inhaler device by a check nut (not shown).

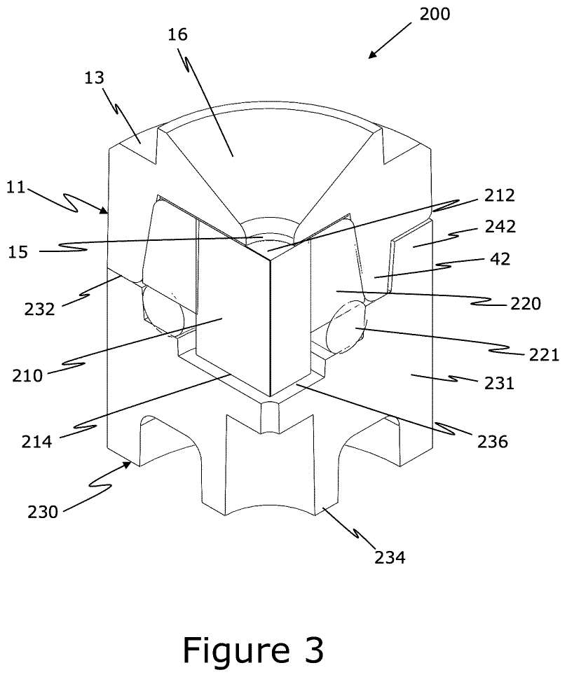

The filter holder 230 also comprises two rotational alignment features 240 (not visible in FIG. 3) and 242, which define projections extending from the first end 232 of the filter holder 230. The rotational alignment features 240, 242 are arranged to interlock with the two rotational alignment features 40, 42 on opposite sides of the first end 12 of the MIM nozzle holder 10 to prevent relative rotation of the MIM nozzle holder 10 with respect to the filter holder 230. The restriction of this relative rotation prevents any twisting forces being applied to the nozzle seal 220 and ensures that the nozzle chip 210 is aligned to the oblong-shaped recess 14 of the MIM nozzle holder 10 and cannot adversely contact the filter holder 230.

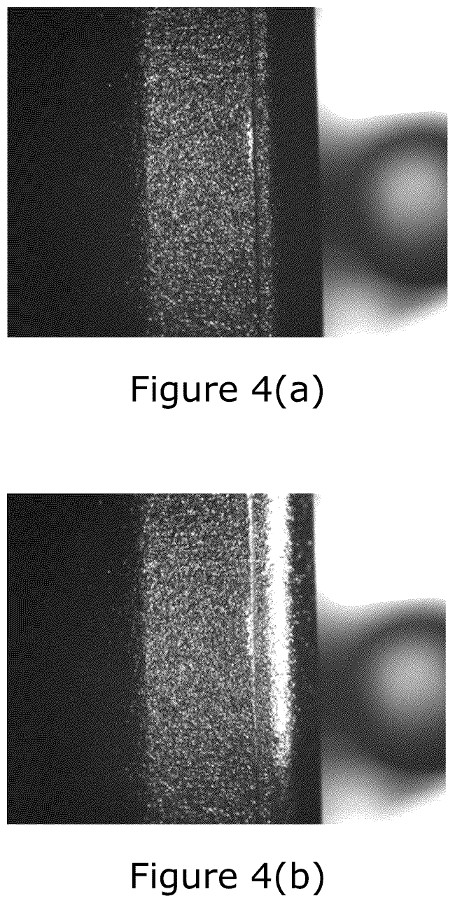

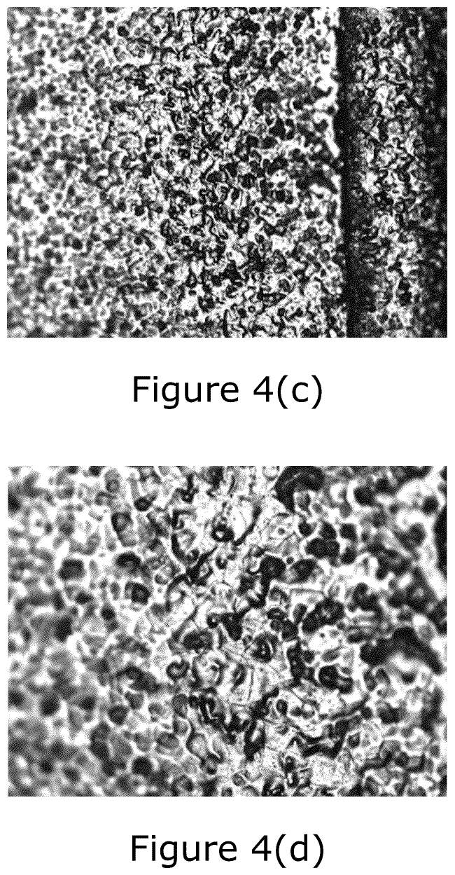

FIG. 4(a) shows the surface of a MIM nozzle holder at 2× magnification. FIG. 4(b) shows the surface of the MIM nozzle holder in a lightbox at 2× magnification. FIG. 4(c) shows the surface of the MIM nozzle holder at 10× magnification. FIG. 4(d) shows the surface of the MIM nozzle holder at 20× magnification.

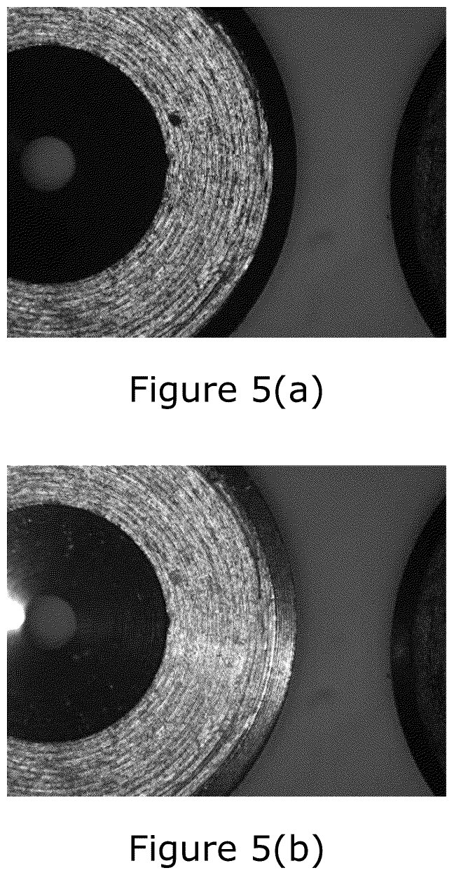

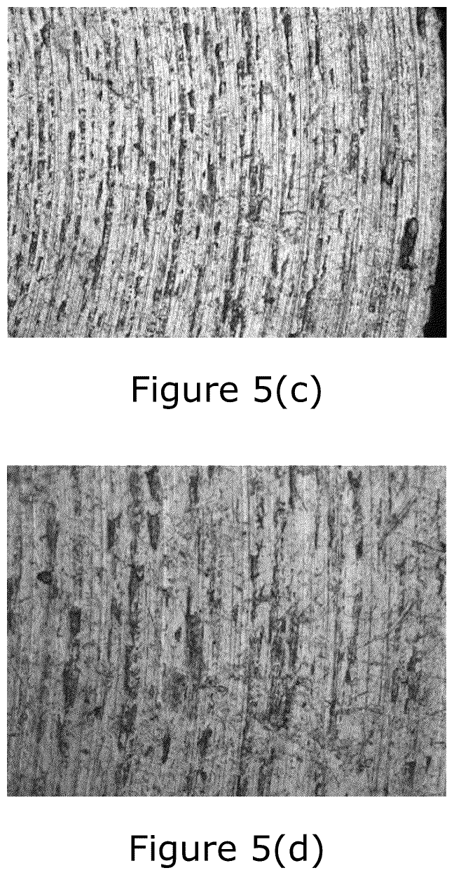

FIG. 5(a) shows the surface of a turned nozzle holder at 2× magnification. FIG. 5(b) shows the surface of the turned nozzle holder in a lightbox at 2× magnification. FIG. 5(c) shows the surface of the turned nozzle holder at 10× magnification. FIG. 5(d) shows the surface of the turned nozzle holder at 20× magnification.

A comparison between FIGS. 4(a) to (d) and FIGS. 5(a) to (d) shows that the surface texture of the MIM nozzle holder is markedly different to the surface texture of the turned nozzle holder. The surface texture of the turned nozzle holder is periodic, showing a repeating pattern of equally spaced apart spaced rings formed during the turning process. By contrast the surface texture of the MIM nozzle holder is non-periodic, defining a random arrangement of pockets of varying shapes and sizes.

### EXAMPLES

Method of Making a MIM Nozzle Holder

Fine stainless steel powder (typically <20 μm particle size) was blended with polyoxymethylene (POM). The stainless steel powder to POM ratio was approximately 60:40 by volume. The blend was placed in a mixer and heated to approximately 80° C. causing the binders to melt. The resulting mass was mechanically mixed until the metal powder particles were uniformly coated with the binders. The mass was cooled and then granulated into free-flowing pellets (feedstock) suitable for use in a metal injection molding machine.

The pelletized feedstock was fed into the injection molding machine where it was heated to approximately 80° C. and injected into a mold cavity under high pressure. The molded part (now termed “green part”) was allowed to cool and then ejected from the mold so the process could be repeated. The tooling can be of multiple cavities for high production rates. The mold cavity was sized approximately 20% larger than the desired size of the nozzle holder in order to compensate for shrinkage that takes place during the subsequent step sintering (detailed below). The shrinkage change is precisely known for each specific material.

Removal of the binder was achieved in several steps whereby the majority was removed before the sintering step, leaving behind only enough binder to handle the parts into the sintering furnace. In this specific example, catalytic debinding in a batch furnace at a temperature of 120° C. in an inert nitrogen atmosphere was used to remove the binder. After binder removal, the part was semi-porous, thereby allowing the remaining binder to easily escape during the sintering process.

The debinded parts were placed on ceramic setters which were loaded into a atmosphere-controlled sintering furnace set to a temperature of 1350° C. in a hydrogen gas atmosphere. The brown parts (i.e. debinded but not yet sintered parts) were slowly heated in a protective atmosphere to drive out the remaining binders. Once the binders were evaporated, the nozzle holder was heated to a temperature of 1350° C. where the void space between the stainless steel particles was reduced or eliminated as the particles fused together. The nozzle holder shrank isotropically to its design dimensions and transformed into a dense solid. The sintered part density is typically around 95-98% theoretical for most materials. The high sintered part density gives the MIM nozzle holder properties that are similar to wrought materials.

Droplet Diameter Analysis

### Comparative Example (not in Accordance with the Invention): Turned Nozzle Holder

A Malvern Panalytical® Spraytec™ laser diffraction system was used to observe the droplet diameters over a number of actuations (i.e. spay events) for an inhalation device comprising a nozzle holder turned from a solid billet. In this example, a high average Dv90 was observed, along with a high variability in the droplet size. The Dv90 value indicates that 90% of the spray volume is contained in droplets that are smaller than the Dv90 value, and 10% is contained in droplets that are larger than the Dv90 value. The high average Dv90 was attributed to droplets forming on the nozzle holder and running back into the path of the central aperture disrupting the spray in subsequent actuations.

### Example 1: MIM Nozzle Holder

A Malvern Panalytical® Spraytec™ laser diffraction system was used to observe the droplet diameters over a number of actuations (i.e. spay events) for the same inhalation device comprising a stainless steel MIM nozzle holder having the same shape, size and surface roughness as the turned nozzle holder used in the comparative example above. In this example, a lower average Dv90 was observed, with less variability in the size of droplets within an actuation and between actuations. The stainless steel MIM nozzle holder was found to reduce the occurrence of droplets running back into the path of the central aperture and therefore reduce disruption to the spray event. This resulted in less variability in the droplet sizes and a reduction in the average Dv90.

1. A nozzle holder for securing a nozzle in an inhalation device, the nozzle holder being obtained or obtainable by metal injection moulding (MIM).

2. A nozzle holder of claim 1, comprising an exterior surface having a water contact angle (θ) equal to or less than 60°.

3. A nozzle holder of claim 1, comprising an exterior surface having a surface roughness average (Ra) of 0.56 microns or more.

4. A nozzle holder of claim 1, wherein the exterior surface of the nozzle holder has a non-periodic surface texture.

5. A nozzle holder of claim 1, wherein 40% or more of the external surface of the nozzle holder is pocketed.

6. A nozzle holder of claim 1, being non-rotationally symmetric.

7. A nozzle holder of claim 1, comprising a non-rotationally symmetric recess, optionally an oblong, elliptical or oval shaped recess.

8. A nozzle holder of claim 1, comprising steel, optionally stainless steel.

9. An inhalation device comprising a nozzle holder according to claim 1.

10. A method of making a nozzle holder according to claim 1, the method comprising the steps of:
mixing metal powder with a polymer binder;
injecting the mixture into a mould with an extruder;
debinding; and
sintering.

11. A method of claim 10, wherein the metal powder comprises steel, optionally stainless steel.

12. A method of claim 10, wherein the polymer binder comprises one or more of polyethylene glycol, polyethylene oxide, polyvinyl alcohol, starches, and/or polyacrylamide.

13. A method of claim 10, wherein the step of debinding comprises one or more of solvent debinding, thermal debinding, catalytic debinding, and/or supercritical debinding.
